(19) United States
(12) Patent Application Publication
(10) Pub. No.: US 2024/0337617 A1
(43) Pub. Date: Oct. 10, 2024

(54) SENSOR ASSEMBLY

(71) Applicant: CLAAS Selbstfahrende Erntemaschinen GmbH, Harsewinkel (DE)

(72) Inventors: Dennis Neitemeier, Lippetal (DE); Christoph Lehmann, BeIm (DE)

(73) Assignee: CLAAS Selbstfahrende Erntemaschinen GmbH, Harsewinkel (DE)

(21) Appl. No.: 18/627,574

(22) Filed: Apr. 5, 2024

(30) Foreign Application Priority Data
Apr. 5, 2023 (DE) DE102023108762.2

(51) Int. Cl. G01N 27/22; A01D 41/127; A01D 43/08; G01N 33/00

(52) U.S. Cl. CPC G01N 27/223; A01D 41/127; A01D 43/085; G01N 33/0098

(57) ABSTRACT

A sensor assembly for detecting at least one material property of a harvested material picked up by a self-propelled harvesting machine and passing through the sensor assembly is disclosed. The sensor assembly includes at least one sensor element which is arranged on a plate-shaped carrier, and an evaluation circuit for evaluating signals which the at least one sensor element generates on the basis of physical contact of the plate-shaped carrier with the harvested material. The plate-shaped carrier is designed as a ceramic-containing plate with a contact surface, which is in contact with the harvested material, and a sensor surface facing away from the contact surface. The at least one sensor element and the evaluation circuit are arranged thereon on the sensor surface of the plate-shaped carrier facing away from the harvested material.

### CROSS REFERENCE TO RELATED APPLICATION

This application claims priority under 35 U.S.C. § 119 to German Patent Application No. DE 10 2023 108 762.2 filed Apr. 5, 2023, the entire disclosure of which is hereby incorporated by reference herein.

### TECHNICAL FIELD

The present invention relates to a sensor assembly, a self-propelled harvesting machine, and a method for manufacturing a sensor assembly.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

EP 3 479 673 B1 discloses a sensor assembly for a self-propelled harvesting machine. To protect the sensor elements arranged on the plate-shaped carrier from the abrasive effect of the harvested material flowing over the sensor elements, the contact surface is provided with a dielectric protective coating. The protective coating comprises a ceramic filler that is bound in a polymer or plastic matrix or a resin.

### BRIEF DESCRIPTION OF THE DRAWINGS

The present application is further described in the detailed description which follows, in reference to the noted drawings by way of non-limiting examples of exemplary embodiment, in which like reference numerals represent similar parts throughout the several views of the drawings, and wherein:

FIG. 1 illustrates a schematic representation of an agricultural harvesting machine designed as a self-propelled forage harvester in a side view.

FIG. 2 illustrates a schematic representation of an agricultural harvester designed as a self-propelled combine harvester in a side view.

FIG. 3 schematically illustrates by way of example a layout of a sensor assembly known from the prior art.

FIG. 4 schematically illustrates an example layout of a sensor assembly according to one aspect of the invention.

### DETAILED DESCRIPTION

As discussed in the background, EP 3 479 673 B1 discloses a sensor assembly for a self-propelled harvesting machine, with a contact surface having a dielectric protective coating. As discussed above, the protective coating comprises a ceramic filler that is bound in a polymer or plastic matrix or a resin. In addition to increasing the distance between the sensor elements and the harvested material by applying the protective coating, the application process may be subject to tolerances, which may result in increased calibration effort. Separately, the moisture absorption of the polymer or plastic matrix or the resin may lead to measurement uncertainties.

Thus, in one or some embodiments, a sensor assembly is disclosed that may be used more flexibly in a self-propelled harvester (interchangeably termed harvesting machine) due to a more space-efficient design.

In one or some embodiments, a sensor assembly is disclosed and configured to detect at least one material property of a harvested material picked up (or collected) by a self-propelled harvesting machine and passing through the sensor assembly. The sensor assembly may include at least one sensor element which is arranged or positioned on a plate-shaped carrier, and an evaluation circuit configured to evaluate signals which the at least one sensor element generates on the basis of physical contact of the carrier with the harvested material. In one or some embodiments, the plate-shaped carrier comprises a ceramic-containing plate with a contact surface that is configured to contact the harvested material and a sensor surface positioned to face away from the contact surface, wherein the at least one sensor element and the evaluation circuit are arranged or positioned thereon on the sensor surface of the carrier facing away from the contact surface (e.g., facing away from the harvested material). The sensor assembly may comprise (or consists only of) a single carrier, which may be mounted (such as mounted directly) in a harvested material flow without requiring the use of a protective coating or any other protective measure. In addition, in one or some embodiments, the sensor assembly is distinguished by a lower installation height which makes the arrangement more flexible and avails more positions for use and options for use. Another advantage of the design of the carrier as a ceramic-containing plate, which may be designed as a ceramic, is the outstanding electrical properties up to high frequency ranges, which may also allow the permittivity to be evaluated in these frequency ranges.

In particular, the at least one sensor element may be designed as at least two electrodes (such as only two electrodes) spaced-apart from one another (e.g., spaced-apart from one another by a predetermined amount). The reduced distance between the crop and the sensor elements designed as electrodes may increase the sensitivity of the sensor assembly, and the resolution for local detection of the permittivity may be increased. The necessary electrodes may be reduced in size, or the requirements for the evaluation electronics may be lowered.

In one or some embodiments, the at least one sensor element and the evaluation circuit may be designed as a printed circuit board. Direct mounting of the at least one sensor element and the evaluation circuit on the carrier may lead to the elimination of bonding steps. By back-casting the carrier on the sensor surface facing away from the contact surface, a direct arrangement in the harvesting machine is contemplated.

In particular, an electrical connection may be arranged or positioned at a distance from the plate-shaped carrier in a sensor housing that may accommodate the plate-shaped carrier in order to connect a cable to the sensor assembly. The plate-shaped carrier may be fixed in the sensor housing in one of several ways, such as by being glued.

Furthermore, in one or some embodiments, a self-propelled harvesting machine is disclosed with the sensor assembly. In particular, a self-propelled harvesting machine is disclosed which is configured for picking up (such as collecting) and processing harvested material using one or more working units arranged or positioned on or in the harvesting machine. The at least one sensor assembly, which may come into physical contact with the harvested material, may be arranged or positioned in the harvesting machine and may be configured to detect at least one harvested material parameter. In one or some embodiments, the at least one sensor assembly is designed according to any of the embodiments disclosed herein.

In one or some embodiments, the at least one sensor assembly is designed as a capacitive sensor assembly.

In particular, the harvesting machine may be designed as a forage harvester or as a combine harvester. While the harvesting machine designed as a forage harvester may process essentially moist harvested material, the harvesting machine designed as a combine harvester may process essentially dry harvested material. In both cases, the arrangement of the at least one sensor assembly, configured to detect at least one harvested material parameter, may be advantageous due to the improved abrasion resistance and the low installation space requirement, such as the low installation height.

In one or some embodiments, the at least one sensor assembly may be arranged or positioned in at least one wall of any one, any combination, or all of: a feed channel; a delivery shaft; or a discharge chute of the forage harvester.

In one or some embodiments, the at least one sensor assembly may be arranged or positioned in at least one wall of any one, any combination, or all of an inclined conveyor, a grain elevator or a return elevator of the combine harvester, and/or the at least one sensor assembly may be assigned to any one, any combination, or all of a threshing device, a separating device or a cleaning device of the combine harvester. In particular, the at least one sensor assembly may be arranged or positioned behind a sieve-like element of the threshing device, the separating device and/or the cleaning device. The sieve-like element may be a threshing concave, a sieve of the separating device, a cleaning sieve, or may be designed as rake-shaped fingers arranged or positioned behind a cleaning sieve.

In particular, the at least one sensor assembly may be configured to detect one or more material properties, such as any one, any combination, or all of: harvested material moisture; harvested material throughput; harvested material density; or lateral distribution. At least for detecting the lateral distribution, a plurality of sensor assemblies may be arranged or positioned, for example, distributed over the width of the separating device or a cleaning sieve behind or below it.

In one or some embodiments, the carrier of the sensor assembly may be integrated in a sensor housing or directly in a surface of one of the working elements coming into contact with the harvested material. A surface of one of the working units coming into contact with the harvested material may be a wall of any one, any combination, or all of: the inclined conveyor; the grain elevator; the return elevator; the feed channel; the delivery shaft; or the discharge chute.

Furthermore, in one or some embodiments, a method for manufacturing a sensor assembly is disclosed. In particular, a method for producing a sensor assembly comprises: producing a plate-shaped carrier comprising (or consisting of) a ceramic material with a contact surface and a sensor surface opposite the contact surface; applying at least one copper layer to the surface of the sensor surface of the plate-shaped carrier by a chemical treatment process; and processing the at least one copper layer to form at least one sensor element and an evaluation circuit.

The sensor assembly manufactured in this way may have the advantage that it enables an arrangement in areas of a self-propelled harvester, which may have been inaccessible previously due to their installation height of sensor assemblies known from the prior art. In particular, the copper layer (in which the at least one sensor element and the evaluation circuit are formed) may be processed, such as by etching or laser ablation.

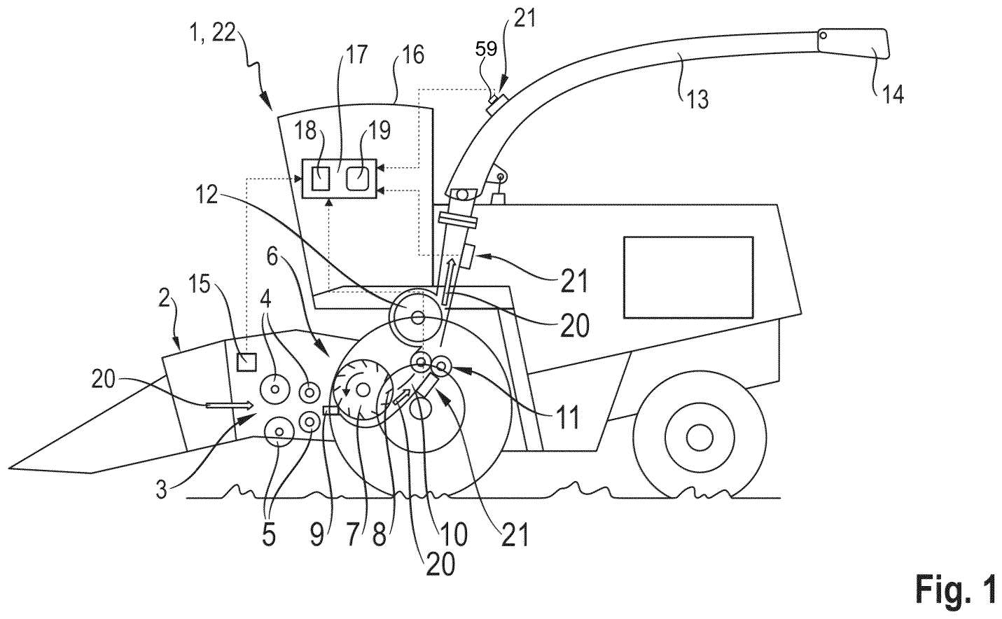

Referring to the figures, FIG. 1 shows a self-propelled harvester 1 designed as a forage harvester 22. Examples of forage harvesters include US Patent Application Publication No. 2022/0071091 A1 and US Patent Application Publication No. 2023/0232740 A1, each of which is incorporated by reference herein in their entirety. The self-propelled harvester 1 may have on which an attachment 2 arranged or positioned in the front area for picking up or collecting harvested material deposited on the ground. The front attachment 2 may vary depending on the type of harvested material to be harvested or picked up. The attachment 2 may pick up the harvested material from the field and may convey it to an intake unit 3, which in the illustrated embodiment comprises (or consists of) a roller group with upper and lower feed rollers 4, 5. The feed rollers 4, 5 of the intake unit 3 may exert a pressing force on the picked up harvested material. The intake unit 3 may convey the harvested material compacted into a harvested material mat to a chopping device 6, which may have a rotatingly-driven cutterhead 7 with chopping blades 8 distributed around its circumference. The chopping blades 8 may cut the harvested material mat fed by the intake unit 3 against a shear bar 9. The cut or chopped harvested material flow 20 may be conveyed by the rotary movement of the cutterhead 7 into a downstream conveying channel 10, from where, depending on the equipment of the forage harvester 22, may be processed by an optional post-processing device 11, such as a conditioning device or corn cracker, arranged or positioned in the crop flow path, and may be conveyed by a downstream, rotationally driven post-acceleration unit 12, additionally accelerated by an adjustable transfer device 13, into an accompanying transport vehicle. The optional post-processing device 11 may either be swung out of the crop flow path or removed completely. The transfer device 13 may be rotated about a vertical axis, for example using a slewing ring. In addition and independently of this, the transfer device 13 may be pivoted about a horizontal axis. A so-called ejection flap 14 may be arranged or positioned at the free end of the transfer device 13, which may be pivoted about a horizontal axis relative to the transfer device 13.

A drive motor may be provided and configured to drive the working units of the harvester 1, e.g., any one, any combination, or all of the attachment 2, intake unit 3, chopping device 6, the post-processing device 11, and the post-acceleration unit 12. The working units may be connected to the motor output shaft of the drive motor by a main drive train (not shown). The drive motor may also serve to operate a hydrodynamic drive of the forage harvester 22.

The intake unit 3 may have a layer height sensor 15. Using the layer height sensor 15, the presence of harvested material and the throughput of harvested material may be determined.

The harvester 1 may comprise a driver's cab 16 in which an input/output unit is arranged or positioned. Furthermore, the harvester 1 may comprise a control device 17, which may comprise a memory unit 18 for storing data and a computing unit 19 for processing the data saved in the memory unit 18. The control device 17 may be configured to support an operator of the harvester 1 in operating it. The control device 17 may also be configured to control the various working units of the harvester 1. Further, the control device 17 may communicate with the at least one sensor assembly 21. In this regard, the control device 17 may be in communication with the one or more working units and the at least one sensor assembly 21.

The computing unit 19 may include at least one processor. In one or some embodiments, the processor may comprise a microprocessor, controller, PLA, or the like. Similarly, the memory unit 18 may comprise any type of storage device (e.g., any type of memory). Though the computing unit 19 and the memory unit 18 are depicted as separate elements, they may be part of a single machine, which includes a microprocessor (or other type of controller) and a memory. Alternatively, the computing unit 19 may rely on the memory unit 18 for all of its memory needs. The memory unit 18 may comprise a tangible computer-readable medium that include software that, when executed by the at least one processor of the computing unit 19 is configured to perform any one, any combination, or all of the functionality described herein regarding any computing device.

The computing unit 19 and the memory unit 18 are merely one example of a computational configuration. Other types of computational configurations are contemplated. For example, all or parts of the implementations may be circuitry that includes a type of controller, including an instruction processor, such as a Central Processing Unit (CPU), microcontroller, or a microprocessor; or as an Application Specific Integrated Circuit (ASIC), Programmable Logic Device (PLD), or Field Programmable Gate Array (FPGA); or as circuitry that includes discrete logic or other circuit components, including analog circuit components, digital circuit components or both; or any combination thereof. The circuitry may include discrete interconnected hardware components or may be combined on a single integrated circuit die, distributed among multiple integrated circuit dies, or implemented in a Multiple Chip Module (MCM) of multiple integrated circuit dies in a common package, as examples.

One or more sensor assemblies 21 may be provided or positioned in the conveyor channel 10 along one or more parts of the path of the material flow 20. In one or some embodiments, the at least one sensor assembly 21 is designed as a capacitive sensor assembly. The at least one sensor assembly 21 may be designed or configured to detect any one, any combination, or all of: a harvested material moisture content; a harvested material throughput; a harvested material density; or a lateral distribution. In this regard, the at least one sensor assembly 21 may be configured to generate sensor data, with the sensor data indicative of one or more material properties of the harvested material. In one or some embodiments, the at least one sensor assembly 21 may transmit (e.g., wired and/or wirelessly) the sensor data to the computing unit 19 in order for the computing unit to determine the one or more material properties of the harvested material. Alternatively, the at least one sensor assembly 21 may be configured to determine the one or more material properties of the harvested material from the sensor data.

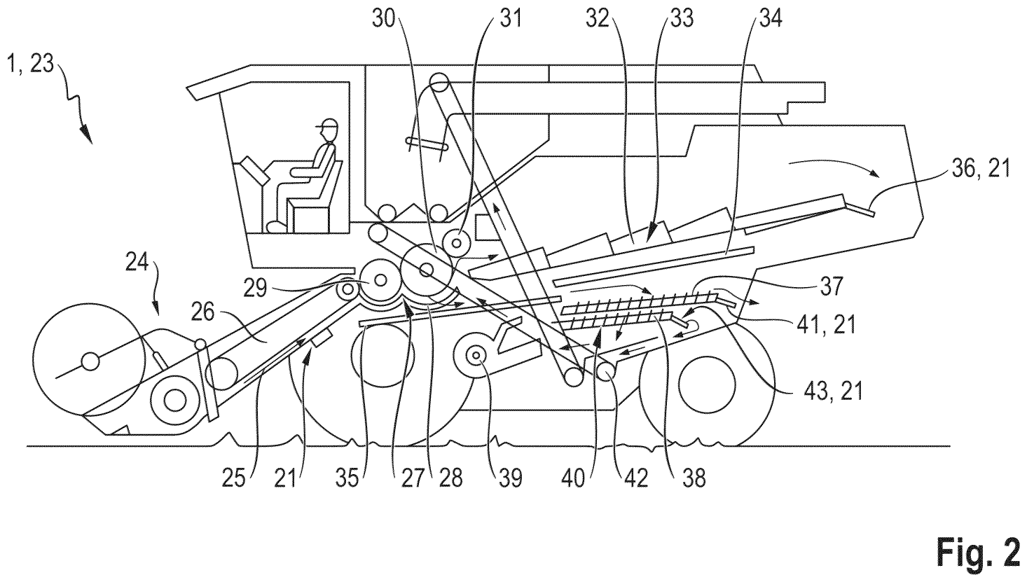

FIG. 2 schematically illustrates a self-propelled harvester 1 designed as a combine harvester 23. Examples of combines include US Patent Application Publication No. 2023/0397533 A1, US Patent Application Publication No. 2024/0090375 A1, US Patent Application Publication No. 2024/0081182 A1, each of which is incorporated by reference herein in their entirety. The combine harvester 1 may have a plurality of working units for conveying and/or processing the harvested material to be harvested (not shown).

In one or some embodiments, the harvested material is picked up by an attachment 24 and fed to a threshing device 27 in a supplied harvested material flow 25 (shown as an arrow) using an inclined conveyor 26. In one or some embodiments, the threshing device 27 may comprise a threshing concave 28, an acceleration drum 29, a threshing drum 30, and a deflection drum 31. A first separation of freely moving grains from the harvested material flow 25 onto a grain pan 35 may occur on the threshing concave 28.

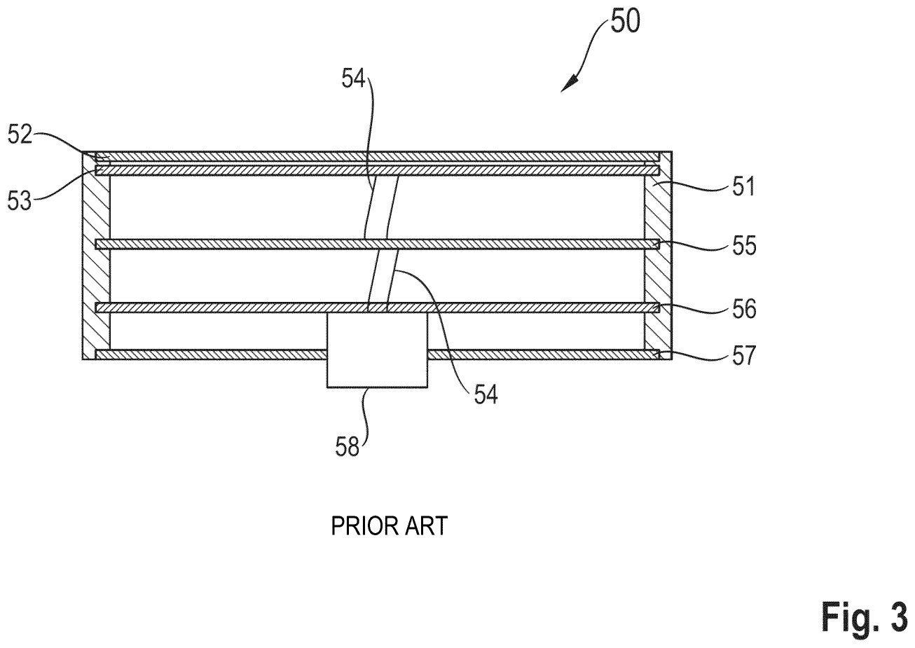

After passing through the threshing device 27, a flow of harvested material exiting therefrom that contains stalk parts and non-threshed grains may be fed to a separating device 33 designed as a straw walker 32. The freely moving grains still contained in the flow of harvested material may be separated using the straw walker 32 in the form of another flow of harvested material to a returns pan 34. A harvested material residual flow coming from the separating device 33, comprising (or consisting primarily of) stalk parts, may be conveyed out of the combine harvester 23. The harvested material residual flow may cross a loss grain counter 36. In one or some embodiments, the loss grain counter 36 is a sensor assembly 21 as shown in FIG. 3. A capacitor, such as a planar interdigital capacitor, may be arranged or positioned on a flat surface in the outlet area of the separating device 33, over which the fourth harvested material flow passes. The sensor assembly 21 may record the elements of the flow of harvested material and may distinguish between grain and non-grain elements.

The combine harvester 23 may have a separating device 33 designed as axial separating rotors instead of the straw walker 32.

Both the harvested material flow exiting the threshing concave 28 and the harvested material flow exiting the separating device 33, which may primarily contain grains, may be combined via the returns pan 34 and the grain pan 35 into a harvested material flow and fed to a cleaning device 40 comprising (or consisting of) a plurality of sieve levels 37, 38 and a blower 39. The grains of this flow of harvested material may be cleaned here and may be separated from non-grain components, such as for example chaff and stalk parts in the form of another flow of harvested material and conveyed out of the combine harvester 23. The further harvested material residual flow may cross another loss grain counter 41, which may also be designed as the sensor assembly 21. The cleaned grain separated by the cleaning device 40 may be conveyed into a grain tank via a grain elevator.

The depicted combine harvester 1 may also have a return auger 42, which may feed a stream of returned harvested material that has been separated by the cleaning device 40 and not completely threshed back to the threshing device 27. On its way to the return auger 42, this stream of returned harvested material may cross a returns grain counter 43, which may also be designed as a sensor assembly 21. From the return auger 42, the returned harvested material flow may be fed to a return elevator, which feeds the returned harvested material to the threshing device 27.

FIG. 3 schematically illustrates by way of example a layout of a sensor assembly 50 known from the prior art. The sensor assembly 50, designed as a capacitive sensor assembly, has a housing 51 in which components of the sensor assembly 50 are arranged or positioned in layers and spatially at a distance from one another. The housing 51 has a contact surface element 52 over which the harvested material flows. The contact surface element 52 consists of an abrasion-resistant material. Printed circuit boards 53 with electrodes are arranged or positioned in the interior of the housing 51 below the contact surface element 52 and at a distance thereto. The electrodes on the printed circuit boards 53 are connected by lines 54 to an evaluation circuit 55 arranged or positioned below the printed circuit boards 53 on another printed circuit board. The printed circuit board with the evaluation circuit 55 is connected by further lines 54 to a plate-shaped plug receptacle 56 located below it. A plug 58, which protrudes from the housing 51, is arranged or positioned on the plug receptacle 56. The underside of the housing 51 is closed by a cover 57. The sensor assembly 50, which consists of a multiple PCB structure, provides a spatial separation of the electrodes, the evaluation circuit 55 and the contact surface element 52 serving for wear protection. This multiple PCB structure results in several disadvantages. For example, the installation space of the sensor assembly 50 is increased. Bonding is required between the various printed circuit boards. Extensive potting or encapsulation of the components of the sensor assembly 50 is necessary. In particular, the distance between the harvested material that flows over the contact surface element 52 and the electrodes on the printed circuit board 53 is large and subject to large assembly tolerances. This leads to an increased calibration effort. Another aspect is that the moisture absorption in the printed circuit boards 53 of the electrodes may lead to measurement uncertainties. Furthermore, voltages between the electrodes and the contact surface element 52 may lead to errors.

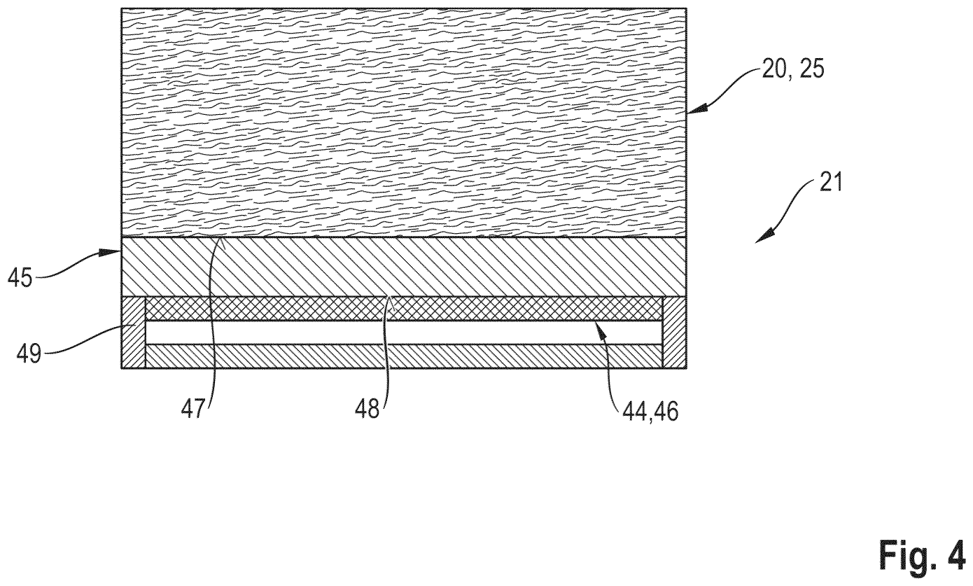

FIG. 4 illustrates a schematic and an example of the layout of the sensor assembly 21. The sensor assembly 21 may comprise at least one sensor element 44, which may be arranged or positioned on a plate-shaped carrier 45, as well as an evaluation circuit 46 configured to evaluate signals, which the at least one sensor element 44 generates (e.g., the sensor data) due to physical contact of the carrier 45 with the harvested material flow 20, 25. For this purpose, the carrier 45 may be designed as a ceramic-containing plate with a contact surface 47, which may be in contact with the harvested material, and a sensor surface 48 facing away from the contact surface 47, wherein the at least one sensor element 44 and/or the evaluation circuit 46 are arranged or positioned thereon on the sensor surface 48 of the carrier 45 facing away from the harvested material. In particular, a technical ceramic may be used as the material for the carrier 45.

The at least one sensor element 44 may be designed as two spaced-apart electrodes. The at least one sensor element 44 and the evaluation circuit 46 may be designed as a printed circuit board. In particular, the at least one sensor element 44 and the evaluation circuit 46 may be integrated into the sensor surface 48 of the 45 carrier (e.g., the at least one sensor element 44 and the evaluation circuit 46 may be fused to and/or built on the sensor surface 48 of the 45 carrier). For this purpose, a copper layer may be applied to the sensor surface 48 of the 45 carrier, in which the at least one sensor element 44 and the evaluation circuit 46 may be formed by a treatment process.

In one or some embodiments, the method for producing the sensor assembly 21 is disclosed. The method may comprise any one, any combination, or all of the following method steps: producing the plate-shaped carrier 45 comprising (or consisting of) a ceramic material with a contact surface 47 and a sensor surface 48 opposite the contact surface 47; applying a metallic layer (such as a copper layer) to the sensor surface 48 of the plate-shaped carrier 45 by a chemical treatment process; and processing the metallic layer (e.g., copper layer) to form the at least one sensor element 44 and/or an evaluation circuit 46.

The copper layer in which the at least one sensor element 44 and the evaluation circuit 46 are formed may be processed by etching or laser ablation.

In one or some embodiments, the reduced distance between the overflowing harvested material and the electrodes of the at least one sensor element 44 may increase the sensitivity of the measuring device, and the resolution may be increased for local detection of the permittivity. The electrodes required for this may be reduced in size, or the requirements for the evaluation electronics may be lowered.

The carrier 45 may be glued into a housing 49. By back-casting the carrier 45 on the sensor surface 48 facing away from the contact surface 47, direct installation in components of the working units of the harvester 1 is also contemplated, which may be inaccessible due to the installation height of sensor assemblies known from the prior art. Due to the generally good, material-dependent thermal properties of the carrier 45, the installation location in the harvester 1 may be subject to only minor restrictions.

An electrical connection 59 (illustrated in FIG. 1) in the form of a plug, a transceiver, or the like may be arranged or positioned at a distance from the plate-shaped carrier 45 in the sensor housing 49 (e.g., within the sensor housing 49 and/or adjacent to the sensor housing) accommodating the carrier 45 in order to connect a cable to the sensor assembly 21. Thus, in one example, the electrical connection 59 may be positioned at a distance from the carrier in and/or on and/or proximate to the sensor housing 49 accommodating the carrier 45 in order to communicate (e.g., wired and/or wirelessly) one or more signals from the sensor assembly to an external electrical device (e.g., the control device 17). Thus, in one embodiment, the electrical connection may be configured to communicate wired and/or wirelessly. This connection is illustrated, for example, in FIG. 1, in which the electrical connection 59 of the sensor assembly 21 is connected (wired via the cable and/or wirelessly transmitting) via arrows to control device 17.

Further, it is intended that the foregoing detailed description be understood as an illustration of selected forms that the invention may take and not as a definition of the invention. It is only the following claims, including all equivalents, that are intended to define the scope of the claimed invention. Further, it should be noted that any aspect of any of the preferred embodiments described herein may be used alone or in combination with one another. Finally, persons skilled in the art will readily recognize that in preferred implementation, some, or all of the steps in the disclosed method are performed using a computer so that the methodology is computer implemented. In such cases, the resulting physical properties model may be downloaded or saved to computer storage.

### LIST OF REFERENCE NUMBERS

1 Harvester 34 Returns pan
2 Attachment 35 Grain pan
3 Intake unit 36 Loss grain counter
4 Feed roller 37 Screening level
5 Feed roller 38 Screening level
6 Chopping device 39 Fan
7 Cutterhead 40 Cleaning device
8 Chopping blade 41 Loss grain counter
9 Shear bar 42 Return auger
10 Conveying channel 43 Loss grain counter
11 Post-processing device 44 Sensor element
12 Post-acceleration unit 45 Carrier
13 Transfer device 46 Evaluation circuit
14 Discharge flap 47 Contact surface
15 Layer height sensor 48 Sensor surface
16 Driver's cab 49 housing
17 Control device 50 Sensor assembly
18 Memory unit 51 housing
19 Computing unit 52 Contact surface element
20 Harvested material flow 53 Printed circuit board
21 Sensor assembly 54 Cable
22 Forage harvester 55 Evaluation circuit
23 Combine harvester 56 Plug receptacle
24 Attachment 57 Cover
25 Harvested material flow 58 Plug
26 Inclined conveyor 59 Electrical connection
27 Threshing device
28 Threshing concave
29 Acceleration drum
30 Threshing drum
31 Deflection drum
32 Straw walker
33 Separating device

1. A sensor assembly configured to detect at least one material property of a harvested material picked up by a self-propelled harvesting machine and passed through the sensor assembly, the sensor assembly comprising:
at least one sensor element positioned on a carrier and configured to generate one or more signals based on physical contact of the carrier with the harvested material;
an evaluation circuit configured to evaluate the one or more signals generated based on the physical contact of the carrier with the harvested material; and
the carrier comprising: (i) a ceramic-containing plate with a contact surface that is configured to contact with the harvested material; and (ii) a sensor surface facing away from the contact surface; and
wherein the at least one sensor element and the evaluation circuit are positioned on the sensor surface of the carrier facing away from the harvested material.

2. The sensor assembly of claim 1, wherein the carrier comprises a plate-shaped carrier.

3. The sensor assembly of claim 1, wherein the at least one sensor element is designed as at least two electrodes spaced apart from one another by at least a predetermined amount.

4. The sensor assembly of claim 1, wherein the at least one sensor element and the evaluation circuit comprise a printed circuit board.

5. The sensor assembly of claim 1, wherein the at least one sensor element and the evaluation circuit are integrated into the sensor surface of the carrier.

6. The sensor assembly of claim 1, wherein an electrical connection is positioned at a distance from the carrier in a sensor housing accommodating the carrier in order to communicate one or more signals from the sensor assembly to an external electrical device.

7. A self-propelled harvesting machine configured to collect and process harvested material using one or more working units positioned on or in the self-propelled harvesting machine, the self-propelled harvesting machine comprising:
the one or more working units;
at least one sensor assembly; and
a control device in communication with the one or more working units and the at least one sensor assembly;
wherein the at least one sensor assembly comprises:
at least one sensor element positioned on a carrier and configured to generate one or more signals based on physical contact of the carrier with the harvested material;
an evaluation circuit configured to evaluate the one or more signals generated based on the physical contact of the carrier with the harvested material; and
the carrier comprising: (i) a ceramic-containing plate with a contact surface that is configured for contact with the harvested material; and (ii) a sensor surface facing away from the contact surface; and
wherein the at least one sensor element and the evaluation circuit are positioned on the sensor surface of the carrier facing away from the harvested material.

8. The self-propelled harvesting machine of claim 7, wherein the carrier comprises a plate-shaped carrier.

9. The self-propelled harvesting machine of claim 7, wherein the at least one sensor assembly comprises a capacitive sensor assembly.

10. The self-propelled harvesting machine of claim 7, wherein the self-propelled harvesting machine comprises a forage harvester or a combine harvester.

11. The self-propelled harvesting machine of claim 10, wherein the at least one sensor assembly is positioned in at least one wall of one or more of a feed channel, a delivery shaft, or a discharge chute of the forage harvester.

12. The self-propelled harvesting machine of claim 10, wherein the at least one sensor assembly is positioned in at least one wall of one or more of an inclined conveyor, a grain elevator, or a return elevator of the combine harvester.

13. The self-propelled harvesting machine of claim 10, wherein the at least one sensor assembly is assigned to one or more of a threshing device, a separating device, or a cleaning device of the combine harvester.

14. The self-propelled harvesting machine of claim 7, wherein the at least one sensor assembly is configured to detect one or more of a harvested material moisture content, a harvested material throughput, a harvested material density or a lateral distribution.

15. The self-propelled harvesting machine of claim 7, wherein the carrier of the sensor assembly is integrated into a sensor housing or directly into a surface of one of the one or more working units coming into contact with the harvested material.

16. A method for producing a sensor assembly comprising:
producing a plate-shaped carrier comprising a ceramic material with a contact surface and a sensor surface opposite the contact surface;
applying a copper layer to at least a part of the sensor surface of the plate-shaped carrier by a chemical treatment process; and
processing the copper layer to form at least one sensor element and an evaluation circuit.

17. The method of claim 16, wherein the plate-shaped carrier consists of the ceramic material with the contact surface and the sensor surface opposite the contact surface.

18. The method of claim 16, wherein the copper layer is applied by etching or laser ablation.
